(19) United States
(12) Patent Application Publication
(10) Pub. No.: US 2006/0198874 A1
(43) Pub. Date: Sep. 7, 2006

(54) Warming and cooling devices made of hypoallergenic organic materials

(76) Inventor: Patsye Stanley, St. Charles, MO (US)

Correspondence Address: Patrick D. Kelly, 11939 Manchester #403, St. Louis, MO, 63131, US

(21) Appl. No.: 11/069,478

(22) Filed: Mar. 1, 2005

(51) Int. Cl. A61N 1/39; A61K 9/70; A61K 36/55

(52) U.S. Cl. 424/443; 424/768; 607/1

(57) ABSTRACT

Therapeutic devices for warming and/or cooling limbs, joints, and body parts, to relieve pain or discomfort, are made of entirely hypo-allergenic materials, such as organically grown cotton fabric and flaxseed. Flaxseed can be heated effectively and safely in a microwave oven, and has a relatively high “specific heat”, allowing it to impart a sustained therapeutic benefit. A suitable quantity of flaxseed is loaded into an envelope or bag, leaving sufficient room to allow the bag to be bent, twisted, wrapped, or draped in a comfortable manner, around a limb, joint, neck, etc. These devices are suited for use by people who suffer from chemical hyper-sensitivity, allergic syndromes, or immune-related problems, and they can reduce the risk that a person who frequently needs comfort for a chronically painful or uncomfortable body part might create or aggravate an allergic reaction or hyper-sensitivity problem.

### FIELD OF THE INVENTION

This invention is in the field of non-prescription medical and therapeutic devices, and relates to reusable devices for warming or cooling various body parts, made of organic materials that have no dyes or pigments, artificial fragrances, herbicide or pesticide residues, or other chemical residues that might provoke allergenic responses in people sensitive to such residues.

### BACKGROUND OF THE INVENTION

It has long been known that under certain conditions and in various people, warming or cooling a portion of the body, the head, or a limb can have beneficial and even medically therapeutic effects. For example, people with arthritis (which can include any condition that involves inflammation or a similar unwanted condition in a joint) or myalgia (which can include any type of chronic or recurrent muscle pain), or who have suffered from an injury or syndrome involving a joint, bone, limb, tendon, etc., often find that warming or cooling the affected joint(s), muscles, or limbs can provide substantial relief (this frequently is done immediately before bedtime, or in bed, to help the person sleep better). Similarly, people who suffer from migraine, cluster, or other recurrent headaches, sinus problems, or other problems involving or affecting the head often obtain comfort and relief by either warming or cooling the face, the back of the head and neck, etc. As a third example, people with neck and shoulder tightness or other discomfort, such as from working for long periods at a computer or other workstation, often find that a warming action provides a substantial and accelerated form of relief, comparable to a neck and shoulder massage. As yet another example, people who are suffering from transient aches or pains (such as a severe toothache that must be endured for hours or even days, before an appointment with a dentist can be obtained) often find that warming or cooling the painful area can at least distract attention away from the pain, thereby making it more bearable. These are merely examples, and various other conditions are known that can benefit from localized warming or cooling.

To provide these types of benefits, various types of warming and/or cooling devices have been created that are designed to be placed in direct contact with the human body (as used herein, “body” includes the limbs and the head, rather than being limited to the chest and abdomen only, sometimes referred to as the “trunk”). As an example of a warming and/or cooling device, classic “hot water bottles” have been available for at least a century (these are not actually “bottles”, since they typically are made of flexible walls of rubber or a similar polymer, causing them to be classified as bladders). Using a “stopper” or cap that engages an opening, the bladder can be filled with either hot or cold water (and with ice cubes or crushed ice, if desired). Typically, the bladder is emptied and the water is discarded, after each use. Since the feeling of hot or cold rubber directly against the skin tends to be uncomfortable, a towel, handkerchief, or similar cloth device is often placed between the rubber and the skin, or the device is pressed against the surface of a piece of clothing.

Although “hot water bottles” are still available, they have largely been superseded by more advanced devices containing gels or similar fluid or semi-fluid materials that can provide two advantages over water. First, the gel or other material that fills such a device will not harden into a solid, when placed in a freezer of the type found in a home. Instead, the gel remains in a viscous and pliable form, thereby allowing the device to be flexed and bent into any desired shape that will accommodate a user. Second, these types of gels usually have “specific heat” levels that are higher than water. “Specific heat” refers to the amount of heating or cooling energy that is required to increase or decrease the temperature of a material, expressed as a number that compares the material to pure water, which has a specific heat of 1.0. Under a fixed set of conditions, a material with a “specific heat” greater than 1.0 will take in or give out more energy, for each degree of temperature change. This allows a gel or similar material with a high specific heat value to stay warm or cool longer under any particular set of conditions, thereby increasing its efficacy and benefits.

However, all such devices, even the most recent ones, have various limitations and shortcomings. For example, most gel-containing devices are limited to use as ice packs, for cooling, and cannot be safely heated in a microwave oven, since they could rupture and pop if heated. Since the gel material in such devices usually is enclosed within a sealed thin-walled plastic envelope that can melt if it touches a hot surface, gel devices generally cannot be safely immersed in a pan of water being heated on a stove, since a spot on the plastic envelope might melt or weaken if it touches a bottom or side wall of a pan on a stove.

Just as importantly, many heating and warming devices cannot be used safely and comfortably by segments of the population who can be described as “hyper-sensitive” to various chemicals and chemical residues. As suggested by the term, such people are unusually prone to unwanted responses to even very low concentrations of various chemicals and chemical residues. Among various people and in varying settings or situations, such responses can range from mild irritation, through noticeable or even painful discomfort, and in some cases even medically threatening responses.

Since these types of hyper-sensitivity response are triggered most commonly by skin contact with a fabric or other material that contains trace quantities of an irritating chemical residue, such responses frequently are manifested initially as rashes, redness, tenderness, a feeling of increased sensitivity or tenderness, or similar symptoms of skin irritation. However, responses among hyper-sensitivity sufferers can also trigger or eventually culminate in other problems, such as increased heartbeat rates, increased blood pressure, dizziness, headaches, and breathing difficulties that can be comparable to (or that may be classified as) asthma attacks. While these types of problems are relatively uncommon and generally affect only a small portion of the general population, there is concern that the rates and risks of chemical hyper-sensitivity among the general population are rising, in a manner comparable to similar, accompanying, and quite probably overlapping increases in diagnosed asthma rates.

It also should be noted that if someone in a family is affected by hyper-sensitivity, it can impose major disruptions and demands on the entire family, since nearly everything in a home that is covered with fabric or fibers (including furniture, bedsheets, carpets, all clothing worn by anyone, etc.), or that is impregnated with chemicals (including paint, veneer, linoleum, etc.), or that has been treated with harsh cleaning solutions or other chemicals that may have left a residue, may need to be removed and replaced by “hypo-allergenic” materials.

Finally, it also should be noted that hyper-sensitivity conditions and syndromes can suddenly and rapidly develop and emerge, without any prior warning, in people who have never suffered from such problems before. This is an unwanted and intensely unpleasant by-product of how the immune system utilizes a system that is referred to in medical terms as an “anamnestic” response (that term is derived from Greek words that mean “not forgetting”). While it is not necessary to fully understand the cellular and genetic processes involved, “anamnestic” responses involve the creation of new variants of immune cells, in response to an infection or other triggering event. The newly-created immune cells contain resequenced specialized gene sequences that will create and pump out brand new types of antibodies, which can bind to and help destroy new types of invading bacteria or viruses that have never previously been encountered by the body that is responding to the new challenge.

“Anamnestic” responses by the immune system are heavily involved in the processes that allow a vaccine (against the viruses that cause polio, flu, chicken pox, etc., or against the bacteria that cause tetanus, measles, etc.) to get the immune system up and running and ready to respond, very rapidly, if a true challenge appears, in the form of invading viruses or bacteria. They also help prevent a person from becoming reinfected, again and again, by the same strain of a cold or other infection.

However, this aspect of the immune system can also pose an ongoing threat to a person, in a manner that can be analogized to a “ticking time bomb”. For example, if someone is stung by a bee or wasp, he or she may have no substantial response, other than a transient local pain followed by a couple of days of tenderness at that spot. This can happen numerous times over the course of a lifetime, with no major crises or even significant events caused by each successive bee or wasp sting. However, each and every time a person is stung by a bee or wasp, some level of risk is created that the person's immune system will become “primed” by that event, and will move into an aggressively defensive mode, just waiting for the next event so it can immediately launch a major counterattack. If that happens, a person who has been stung any number of times previously, but who never previously responded in a way that might suggest that serious trouble was approaching, might suddenly launch into a life-threatening response. This type of response is often called an “anaphylactic” response (such as “anaphylactic shock”), where “anaphylactic” is derived from Greek words that translate into “the opposite of protective”. The victim can suddenly become unable to breathe, unable to call for help, and unable to walk or crawl to another location where he or she will be found quickly and rescued. Hundreds or even thousands of people die every year from insect bites and stings; and, since most people who suffer from a known life-threatening hyper-sensitivity to insect stings or bites usually carry potent injectable anti-histamines or other protective drugs whenever they go outdoors, the majority of people who die from such reactions had no advance warning of any sort, suggesting that they were in danger and needed to be especially vigilant against a slowly-developing hyper-sensitivity response that they did not know was approaching.

When that same mechanism, operating principle, and cautionary realization about how the immune system actually works is taken into account in the context of home furnishings and medical devices, it leads intelligent people to realize that they may well be better off and subject to fewer risks. over the long run, if they will take rational steps to limit their exposure to unnecessary, unwanted, and potentially toxic or allergenic chemical residues. This does not arise from “Luddite” philosophy or politics, by people opposed to any technology or progress; instead, it arises from a realistic and balanced study of how a mammalian immune system actually works, and how it sometimes can be hijacked and diverted in ways that can trigger autoimmune, allergenic, and hyper-sensitivity disorders.

It must also be recognized that hyper-sensitivity syndromes and similar disorders tend to arise, much more frequently, among the same people who are already suffering from the types of discomforts and disorders that lead them to need heating or cooling packs on a chronic and/or frequent basis. Again, the reasons for this correlation arise from certain aspects of how the immune system functions, in mammals. To help non-specialists understand the correlations and overlaps between chronic pain, and chemical hyper-sensitivity, an analogy can be used, involving police who must work in high-crime areas, such as impoverished inner city neighborhoods where drug use and public drunkenness are endemic. If high concentrations of police and police cars are already targeting a high-crime area, ready to respond rapidly to any disturbance and generally having an attitude of not tolerating insults or provocations, it will tend to increase the numbers of arrests made in such areas.

In an analogous manner, if a particular part of the body is already suffering from chronic inflammation, soreness, or other distress, the odds increase substantially that the immune system will respond by placing more “police” (i.e., immune cells) in that particular part of the body. In addition, those immune cells will tend to be in a state of heightened readiness, so they can respond rapidly and strongly to any insults or provocations. This is exactly the type of “loaded, primed, and cocked” condition that can create and then amplify an overly aggressive response to a minor provocation that otherwise might have gone unnoticed.

As a result, a limb, joint, or other part of the body that is suffering from chronic or recurrent pain, of a type that needs frequent or chronic use of heating or cooling pads, becomes an area of elevated risk. Because of how the immune system works, that particular site can become the “launch site” for an immune system response that can develop into a serious or severe case of chemical hyper-sensitivity.

People who understand these types of factors and risks, and who understand how the immune system can expand and increase a chronic or recurrent pain problem into a larger problem or major crisis involving chemical hyper-sensitivity, have realized that, if they chronically or frequently need a heating or cooling pad, then they would well-advised to use one that qualifies as “hypo-allergenic”.

It should also be recognized that people who suffer from chemical hyper-sensitivity disorders tend to have increased needs for warming or cooling devices that can be placed against aching body parts. Regrettably, chemical hyper-sensitivity disorders frequently create (or are accompanied by) the same types of aches, pains, and discomforts that often require heating or cooling treatments. As a direct result, people who suffer from chemical hyper-sensitivity tend to be frequent and recurrent users of the types of discomfort-relieving devices described herein, and especially need these specialized types of hypo-allergenic devices.

It is also worth noting that the prefix hypo- has the opposite meaning of hyper-, and the two prefixes can be confusing, since they look and sound similar to each other. The prefix hypo- indicates below, or less than. For example, a hypodermic needle penetrates the skin and goes beneath and below the skin, and hypothermia occurs when someone's body temperature goes below the normal range. By contrast, hyper- means the opposite, and refers to unusually high, or excessive. A hyperactive child is unable to control his or her impulses, and chemical hypersensitivity refers to unwanted and excessive sensitivity to one or more chemicals.

Accordingly, hypo-allergenic materials include materials that have been grown, manufactured, or otherwise processed in ways that totally avoid, or that minimize to the greatest extent possible, any exposure to, treatment by, or content of, any chemicals or chemical residues that are not absolutely necessary, or that fall within various categories that are known to cause problems in some segments of the population (including herbicides, pesticides, non-natural pigments, perfumes or fragrances, etc.).

Specialized segments of industry and commerce have arisen that specialize in making and supplying materials that truly and properly qualify as hypoallergenic materials, even in the eyes of “purists” who are deeply and personally concerned about chemicals and chemical residues because of personal or family hyper-sensitivity problems. Although many suppliers and sellers claim to offer such materials, such claims are not always reliable, and the best way to identify truly reliable suppliers of organic and hypo-allergenic materials is by: (i) obtaining an independent and impartial confirmation from a trade group, such as the Organic Trade Association, which owns and runs the website www.ota.com, and (ii) obtaining a written guarantee, from the supplier, that the materials being purchased are indeed certified as being organic and/or hypo-allergenic.

Since flaxseed and cotton fabric are used in the products described below, it should be noted that fully organic, hypo-allergenic flaxseed is sold by the Caudill Seed Company (www.caudillseed.com), the supplier of the hypo-allergenic flaxseed used by the Applicant herein. In addition, the Applicant purchased and used organic and hypo-allergenic cotton fabric from the Vreseis Company (www.vreseis.com).

Accordingly, one object of this invention is to disclose and provide devices that can be used interchangeably for either warming or cooling a body part (such as a limb, joint, head or neck area, or chest or abdominal area), wherein such devices are made entirely of hypo-allergenic materials.

Another object of this invention is to disclose and provide warming and/or cooling devices made entirely of hypo-allergenic materials, for use by patients who already suffer from known chemical hyper-sensitivity disorders.

Another object of this invention is to disclose and provide warming and/or cooling devices made entirely of hypo-allergenic materials, to minimize the risk that frequent use of such a device on one or more body parts that are already suffering from chronic or recurrent problems will increase the risk that a chemical hyper-sensitivity condition will develop in the user.

These and other objects of the invention will become more apparent through the following summary, drawings, and detailed description.

### SUMMARY OF THE INVENTION

Warming and/or cooling devices for use in relieving pain or discomfort are made of entirely hypo-allergenic materials. Such materials include cotton fabric or similar natural fabrics, which are sewn into a generally elongated and tubular envelope or bag, and a granular material that can be heated effectively in a microwave oven and that has a relatively high specific heat value, such as flax seed. A suitable quantity of the granular material is loaded into the envelope or bag, leaving sufficient empty space in the bag to allow the bag to be bent, twisted, wrapped, draped, etc. in a comfortable manner, around a limb, joint, neck, etc. Both the fabric and the granular material must be grown, manufactured, and processed with no pesticides, synthetic fertilizers, artificial pigments, or similar chemicals, and they must be processed using machinery that does not contain any chemical residues from processing chemically-treated plants or materials. Such devices are well-suited for use by people who suffer from chemical hyper-sensitivity, allergic syndromes, or immune-related problems. Such devices also can reduce the risk that a person who frequently uses a warming and/or cooling device to provide comfort to a chronically or frequently painful or uncomfortable body part might create or aggravate an allergic reaction, chemical hypersensitivity problem, or similar problem.

### BRIEF DESCRIPTION OF THE DRAWING

FIG. 1 is a perspective view of a hypoallergenic bag, made of entirely organic, pesticide-free, pigment-free cotton fiber, partially filled with flax seed, also grown entirely organically and with no pesticides.

### DETAILED DESCRIPTION

As briefly summarized above, this invention relates to devices that can both warm and cool (at different times) a body part (as used herein, any reference to “body part” is not limited to the trunk, and includes a limb, joint, head, neck, etc.).

These types of warming and cooling devices can be used to soothe and comfort various aches, pains, and other annoyances; when chilled, they also can reduce the swelling that accompanies an injury such as a sprain. In many cases these devices can provide substantial medical or other therapeutic benefits (such as providing substantial relief for discomfort caused by arthritis or various muscular or bone problems. Accordingly, although these devices can be purchased without requiring any type of prescription or approval, they frequently are recommended by physicians, physical or other therapists, chiropractors, etc. Since these devices have known and recognized medical uses, they are classified and registered by the U.S. Food and Drug Administration as “Class 1” medical devices. As such, they can be sold over-the-counter or via Internet or mail-order sales, without requiring a prescription from a physician.

Their mode of use is relatively simple and straightforward, and involves either heating or cooling the device (depending on the desires of a user at a particular time), then placing it against the surface of a selected body part, either directly, or with a piece of clothing or fabric between the device and the user's skin. If desired, they also can be used as bed-warmers, or for similar purposes; for example, if a person suffers from a neuralgic or neuropathic skin or muscle condition that is aggravated by cold, that person can place one of these devices, which has been warmed up, into a laundry hamper or other suitable container, with a set of clothes that will be put on that day. Within a few minutes, the device will warm up the clothes, allowing the person to put on the clothes without aggravating the pain condition.

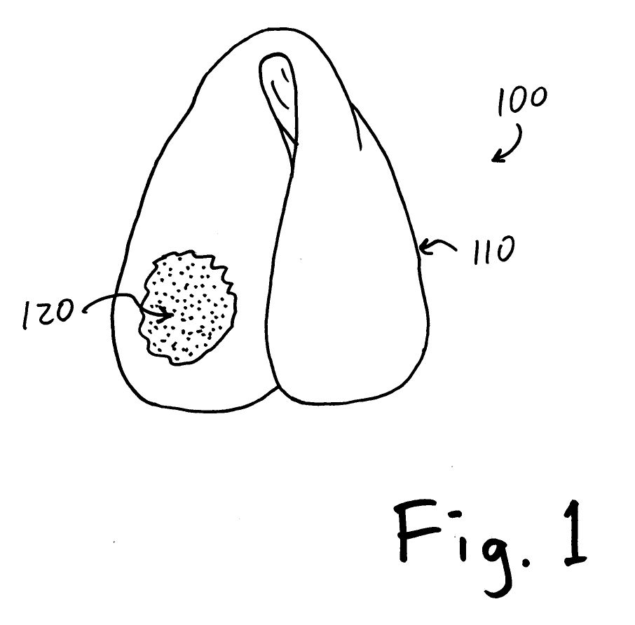

Placement of the device against or around a selected body part, in a manner that warms or cools a skin region as well as tissues or joints beneath the surface area, can be done in any suitable manner, and takes advantage of the fact that, in a preferred embodiment, the device is manufactured in the form of an elongated tube (alternate configurations can be made if desired, such as in the shape of a teddy bear or other stuffed animal). The bag (which can also be called an envelope, enclosure, or similar terms), is made of a flexible fabric, such as cotton. It is filled with flaxseed to only a fraction of its capacity; a filled bag usually is only about ⅔ to ¾ full, depending on the size and weight of the bag, as indicated by the configuration in FIG. 1. This renders a tubular bag easily and readily flexible, so it can be draped across or wrapped around neck, limb, joint, etc., in a manner that allows conformance to the surface being covered, leading to a practical level of stability. When used in a draping-type placement (such as around the neck, across a shoulder, etc.), the user can stand up and walk around if desired, while carrying the device. Alternately, a user can lay, sit, or otherwise rest on a recliner, bed, sofa, or other piece of furniture, carpet, etc., which will support most of the weight of the device while it is being used.

Because these devices will be placed in contact with skin surfaces (including, in many cases, surfaces of body parts that are uncomfortable, tender, or aching), any temperatures that will be of practical interest must be within a relatively limited range. Accordingly, terms indicating elevated temperatures (such as warm or warmed, heating, hot, etc.) are used interchangeably, and refer to surface temperatures greater than internal body temperatures (about 370 centigrade, or 98° Fahrenheit), up to a practical maximum of roughly 115 to 125° F. (temperatures above that level are too hot for desirable use, and many users will not want to go that high). Terms such as cool, cold, chilled, etc., also are used interchangeably with each other, and refer to temperatures lower than about 20° C. (about 70° F.), which is about equal to typical skin temperature on the exposed portion of a limb (i.e., not including underarm or groin areas, which have higher skin temperatures) when a person is in a comfortable room that is in the low 70's F.

In everyday use, a warming or cooling device as disclosed herein can be heated quickly and conveniently, by placing it in a microwave oven and heating it until it reaches a desired temperature. While preferred heating times will vary (due to variations in microwave oven power), a good a fairly reliable rule of thumb is that a device should be heated in a microwave for one minute, for each pound of flaxseed. Accordingly, a device with 2 pounds of flaxseed generally should be heated for about 2 minutes. The device should not be heated excessively, to a point where it becomes too hot to lift and handle; otherwise, the flax seeds inside the device will lose their internal moisture and oils at accelerated rates, and will not last as long as can be achieved by proper and gentle heating. If desired, a device can be removed from a microwave oven, during heating, and briefly massaged or manipulated for a few seconds, to mix together and redistribute the warmer and cooler seeds in the bag in a manner that will “even out” any hot spots. However, that usually is not necessary, in most microwave ovens.

For chilling or cooling purposes, a device as disclosed herein can be placed inside any conventional refrigerator or freezer, for an indefinite period of time. If chilled in a freezer, it usually will be preferable to place a towel or other piece of fabric between the device and the skin, to avoid unpleasant sensations of intense cold. However, in any situation where an ice pack would be appropriate (such as to reduce swelling after a sprain, or to minimize skin damage and pain if a person is burned by a hot stove or oven), a device that was chilled in a freezer can be applied directly to the skin.

It should be noted that one of the advantages of warming and cooling devices that contain flax seed or other granular materials is that they can be heated whenever desired, and chilled whenever desired. This is in contrast to devices that contain gels or other liquids, which can be easily chilled in any conventional refrigerator or freezer, but which pose a risk of bursting if heated improperly. Such devices can be heated only with great caution, and generally cannot be heated safely using a microwave oven.

Accordingly, various claims below refer to an ability to be safely heated or cooled. Any reference to safety, in any claim, relates and refers to the ability to safely and conveniently heat a device in a microwave oven, in a manner that will not and cannot create any risk of bursting and rupturing, even if a device is severely overheated. This claim language avoids and excludes devices that contain a liquid, gel, paste, or similar material that is sealed inside a water-tight and pressure-tight envelope, since devices pose a substantial risk of bursting, if inadvertently overheated in a microwave oven.

Any reference herein to refrigerator or freezer is limited to conventional refrigerators or freezers, as used for storing food. It does not include specialized freezers of the type that can achieve super-cold (“cryogenic”) temperatures for working with compounds such as dry ice, liquid nitrogen, etc.

With regard to claim limitations that refer to “warming and cooling”, it should be clearly understood that only one operation (either warming, or cooling) can be performed at any given time. The point that merits attention is that unlike gel-containing or other devices of the prior art, which can only be used safely and conveniently for cooling, a single device as disclosed herein can be used easily and conveniently for heating when desired, and for cooling when desired.

An important aspect of this invention is that the devices disclosed herein must be made entirely of special, carefully-created, carefully-selected hypo-allergenic materials. This is crucial, since a substantial portion of any sales of such devices will be to people who have chronic aches, pains, and other problems because they suffer from either or both of two conditions: (i) they suffer from a condition of elevated sensitivity to chemical residues, and/or (ii) they need therapeutic devices that can provide comfort to specific body regions because they're suffering from chronic discomfort, swelling, or irritation of a type that indicates their immune system may have reached “hair-trigger” status, where even a minor triggering event may provoke an aggressive counterattack by the immune system, which could lead to severe problems such as a full-blown autoimmune disorder or chemical hyper-sensitivity syndrome.

It must be recognized that the types of carefully-created, carefully-selected hypo-allergenic fabrics and seeds that are required for the devices disclosed herein are much, much more expensive than comparable candidate materials that otherwise could be used to make devices that would appear to be equivalent to the devices disclosed herein. Organically-grown, hypo-allergenic fabrics and seeds that are totally free of even trace amounts of chemical residues are very expensive; indeed, their high costs can even be startling, to people who have never looked into pricing for such items. Those increased costs would initially appear to be unreasonable, unless someone recognizes and understands both: (i) the additional expenses, complications, and burdens of trying to grow and manufacture things in ways that are outside the mainstream of American commerce; and, (ii) the crucial importance of hypo-allergenic fabrics and seeds, in a relatively small segment of the population that needs such materials to avoid (or to help them cope with) serious and even life-threatening medical problems.

Because it can be sold at unusually high prices (even if only in relatively small quantities to a limited segment of the population), organically-grown and residue-free hypo-allergenic cotton fabric is available from certain specialty suppliers, such as the Vreseis Company (www.vreseis.com). By selecting, cultivating, and hybridizing certain strains of cotton that initially showed traces of natural coloring, that company has created several types of cotton and cotton fabric that are naturally pigmented (including but not limited to the beige and/or light brown coloration that is fairly common in various types of non-bleached organic materials). These naturally pigmented materials are especially useful and desirable for skin-contacting devices as disclosed herein, since ordinary use over a span of weeks or months will usually lead to some degree of soiling and darkening, which becomes substantially more noticeable and unappealing when it occurs against a background of bright white. Accordingly, one aspect of this invention comprises warming or cooling devices use naturally-pigmented cotton fabrics to provide the bag (or envelope, enclosure, etc.), since such devices can retain their attractiveness for longer periods of time than devices that have a stark white color that will tend to emphasize and call attention to any smudges or soiling.

Similarly, organically-grown, residue-free hypo-allergenic flaxseed is available from specialty suppliers, such as the Caudill Seed Company (www.caudillseed.com). The selection and preferred use of flaxseed (rather than other types of seeds, grains, or granular material) for use as described herein deserves attention. Flaxseed are well-suited for such use, because of a combination of three important traits.

First, flaxseed contains enough moisture (water) content to enable it to be heated effectively in a microwave oven. In general, microwave ovens used in homes have been “tuned” to use microwave frequencies that are maximally absorbed by water, since some concentration of water is present in the large majority of foods that people wish to heat in such ovens. However, because high levels of moisture content tend to render any food source much more susceptible to attack and spoilage by bacteria, mold, and other microbes, most seeds and grains from plants have evolved over time in ways that sharply reduce their moisture content, as the seeds or grains approach maturity and release, by the plant. As it turns out, flaxseed happen to have a moisture content (usually in a range of about 5 to 8%, by weight) that provides them with good resistance against microbial attack, even over a span of months or years, while also enabling them to be heated effectively, rapidly, and conveniently in a microwave oven.

Certain claims refer to “granular material . . . that can be heated effectively in a microwave oven”. Any reference to “effective” heating, in a microwave oven, requires that (i) the granular material must warm up in a relatively even and consistent manner, within a relatively brief and practical timespan, when heated by a conventional microwave oven of the type found in homes and used to heat food. To provide a numerical standard that can be used as a “benchmark” level to determine whether a candidate material can be heated “effectively” in a microwave oven, the following standard is used herein: a granular material can be heated “effectively” in a conventional home-type microwave oven if at least 80% of a one-pound load of such material, placed in a cup or bowl rather than spread evenly across a flat sheet, will be heated by at least 100 Fahrenheit, within two minutes after heating commences, when the oven is set on a high-power setting.

A second trait of flaxseed that renders it highly useful for use as described herein is its relatively high content of oil. Flaxseed contains an unusually high concentration of “essential fatty acids”, which are nutritious oils, including both omega-3 fatty acids (components of oils found mainly in fish) and omega-6 fatty acids (components of many types of “vegetable oils”, usually obtained from seeds or grain such as corn, canola, soybeans, peanuts, etc.). When eaten, these oils are nutritious, but clearly, that is not their intended use when loaded into a warming or cooling device that will be pressed against the skin. Instead, the value of the high oil content in flaxseed, in a warming or cooling device, is that after the device has been chilled or warmed, using a microwave oven (for heating) or a refrigerator or freezer (for chilling), the oils inside the flaxseed in the device will sustain useful therapeutic temperatures for relatively long periods of time. Stated in technical terms, as described in the Background section, flaxseed has a high “specific heat” level, due to its high oil content.

The third trait of flaxseed that renders it highly useful for use as described herein involves the sizes of the seeds (or grains, kernels, etc.). Grains of flaxseed have dimensions that render them ideally suited for use inside bags made of a porous and water-permeable fabric such as cotton. Very tiny grains (comparable to flour or similar powders), and preparations that contain non-homogenous particle sizes (such as materials created by milling operations), tend to allow small quantities of material to permeate out through the fabric enclosure, release unwanted powdery materials that would soil clothes, furniture, carpets, etc. Conversely, larger grains (such as soybeans, corn kernels, beans, etc.) have an irregular “bean-bag” texture, which becomes uncomfortable when applied to a sensitive or painful area, and which can create small “hot spots” when heated kernels are pressed against a skin surface. By contrast, a flaxseed preparation has a consistent and ideal grain size, since the seeds are: (i) small enough to comply closely and comfortably with a skin surface, with no bumpy or irregular feel and no “hot spots”, and (ii) large enough to stay inside a cotton bag without allowing any grains or powdery residue to escape.

Accordingly, FIG. 1 is a perspective view depicting a warming device 100, comprising a fabric bag 110 (which can also be called an enclosure, envelope, etc.), which is filled to roughly a ⅔ or ¾ (by volume) quantity of flaxseed 120. Both the fabric bag 110 and the flaxseed 120 are made of organically grown and hypo-allergenic materials, as discussed above. The bag or enclosure must be made of a fabric or similar material that is permeable to water, to allow it to be heated safely in a microwave oven without risk of bursting due to steam pressure; however, the fabric must also be able to safely and reliably retain flaxseed (or other selected hypo-allergenic material) within the bag, without allowing substantial quantities of seeds or of grit, powder, or similar material to escape from the bag, creating unwanted residues.

To allow the device to remain adequately flexible, in a way that allows it to be shaped and manipulated in ways that allow it to conform to an irregular surface, the bag must not be overfilled with the granular material. For relatively small bags, such as a 1.5 pound device, levels approaching 90% (expressed as a percentage of the “fully stuffed” potential volume) should not be exceeded, and levels of 70 to 80% are preferred. For larger bags having generally tubular shapes, fill levels of about 80% should not be exceeded, and levels of about 65 to 75% are preferred.

The bag should be sewn together using thread that also is hypo-allergenic; however, it does not need to be natural or organically-grown thread. For example, commercial grades of non-pigmented synthetic thread can be made from selected types of polyester, nylon, or other synthetic materials that are essentially chemically inert for all practical purposes. Such threads will not leach out or otherwise release chemicals that can provoke allergic reactions, especially when thread segments are sewn and used in ways that will minimize any contact with a skin surface. For example, a roughly tubular or cylindrical bag (or indeed a bag having nearly any desired size and shape) can be created by sewing a single continuous first seam around the lateral side edge and one end edge of a piece of fabric that has been folded in half. The mostly-closed bag is then turned inside out, effectively turning it into an elongated pouch with the first seam hidden entirely inside the bag, where it cannot contact any skin surface. The bag is then loaded with an appropriate quantity of flaxseed, and a final short seam is sewn across the short end of the filled bag.

A range of convenient and appropriate sizes can be made and sold. For example, a large device that holds about 3 pounds of flaxseed can be made with dimensions of about 5 by 24 inches; a medium-sized device that holds about 2 pounds of flaxseed can be made with dimensions of about 4 by 20 inches; and a small bag, ideally suited for facial, sinus, or similar use, holding about 1 to 1.5 pounds of flaxseed, can be made with dimensions of about 6 by 10 inches.

Thus, there has been shown and described a new and useful type of warming and cooling device, made entirely of hypo-allergenic materials, for therapeutic and medical use by people who use such devices to help provide relief for various types of aches, discomforts, and other problems. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A device for warming and cooling body parts, comprising:
a. a flexible enclosure made of a hypo-allergenic material that is permeable to water but that will retain a selected hypo-allergenic granular material inside the enclosure;
b. a hypo-allergenic granular material contained within the enclosure, wherein the hypo-allergenic granular material can be heated safely and effectively in a microwave oven.

2. The device of claim 1, wherein the flexible enclosure comprises organically-grown cotton fabric.

3. The device of claim 2, wherein the flexible enclosure comprises fabric that is certified to be free of herbicide, pesticide, pigment, or fragrance chemical residues.

4. The device of claim 1 wherein the hypo-allergenic granular material comprises seeds.

5. The device of claim 4 wherein the hypo-allergenic granular material comprises seeds that are certified as organically grown.

6. The device of claim 5 wherein the hypo-allergenic granular material comprises seeds that are certified to be free of herbicide, pesticide, pigment, or fragrance chemical residues.

7. The device of claim 4 wherein the seeds comprise flax seeds.

8. The device of claim 4 wherein the seeds comprise flax seeds that are certified as organically grown.

9. The device of claim 4 wherein the seeds comprise flax seeds that are certified to be free of herbicide, pesticide, pigment, or fragrance chemical residues.

10. The device of claim 1 wherein not more than about 80 percent of the flexible enclosure's volume has been filled with the granular material, thereby allowing the flexible enclosure to be shaped and manipulated in ways that allow it to conform to an irregular surface.

11. A therapeutic device comprising:
a. a flexible enclosure made of a hypo-allergenic fabric comprising a natural fiber that has been organically grown; and,
b. a hypo-allergenic granular material contained within the enclosure, wherein the granular material is certified to be free of herbicide, pesticide, pigment, or fragrance chemical residues, and wherein the granular material can be heated effectively in a microwave oven.

12. The device of claim 11 wherein the natural fiber comprises cotton.

13. The device of claim 11 wherein the granular material comprise seeds.

14. The device of claim 11 wherein the granular material comprise flax seeds.

15. The device of claim 11 wherein not more than about 80 percent of the flexible enclosure's volume has been filled with the granular material, thereby allowing the flexible enclosure to be shaped and manipulated in ways that allow it to conform to an irregular surface.
